SEQUENCE LISTING (14 sequences)

SEQ ID NO: 1 - protein, 5 aa, Artificial
  Linker Sequence
CXWXC

SEQ ID NO: 2 - protein, 12 aa, Artificial
  Linker Sequence
KXXXXWXCXXXC

SEQ ID NO: 3 - protein, 8 aa, Artificial
  Linker Sequence
KXXXXWXC

SEQ ID NO: 4 - protein, 3 aa, Artificial
  Linker Sequence
CXX

SEQ ID NO: 5 - protein, 3 aa, Artificial
  Linker Sequence
CXW

SEQ ID NO: 6 - protein, 10 aa, Artificial
  Linker Sequence
KXXXXWXCCX

SEQ ID NO: 7 - protein, 4 aa, Artificial
  Linker Sequence
CCXW

SEQ ID NO: 8 - protein, 5 aa, Artificial
  Linker Sequence
CGGGC

SEQ ID NO: 9 - protein, 12 aa, Artificial
  Linker Sequence
KAMHAWGCGGGC

SEQ ID NO: 10 - protein, 8 aa, Artificial
  Linker Sequence
KAMHAWGC

SEQ ID NO: 11 - protein, 3 aa, Artificial
  Linker Sequence
CGG

SEQ ID NO: 12 - protein, 3 aa, Artificial
  Linker Sequence
CXX

SEQ ID NO: 13 - protein, 11 aa, Artificial
  Linker sequence
KXXXWXCGGGC

SEQ ID NO: 14 - protein, 12 aa, Artificial
  Linker sequence
KXXXXWXCGGGC

(19) United States
(12) Patent Application Publication
(10) Pub. No.: US 2010/0216969 A1
(43) Pub. Date: Aug. 26, 2010

(54) IMMOBILISATION OF POLYPEPTIDES BY IRRADIATION

(75) Inventors: Torben Snabe, Hadsten (DK); Maria Teresa Neves-Petersen, Aalborg (DK); Steffen Bjorn Petersen, Aalborg (DK)

Correspondence Address: DARBY & DARBY P.C., P.O. BOX 770, Church Street Station, New York, NY, 10008-0770, US

(73) Assignee: BIONANOPHOTNICS A/S, COPENHAGEN K (DK)

(21) Appl. No.: 11/993,168

(22) PCT Filed: Jun. 27, 2006
(86) PCT No.: PCT/DK06/00377

§ 371 Date: Sep. 22, 2008

Related U.S. Application Data
(60) Provisional application No. 60/697,173, filed on Jul. 6, 2005.

(30) Foreign Application Priority Data
Jun. 27, 2005 (DK) PA2005,00956

(51) Int. Cl. C07K 7/50; C07K 1/00; C07K 7/06

(52) U.S. Cl. 530/317; 530/345; 530/327

(57) ABSTRACT

The present invention relates generally to methods and carriers for cross-linking or immobilising biomolecules such as polypeptides. In particular, the present invention relates to methods and carriers useful for coupling a polypeptide to a carrier via at least one disulphide bond. The carrier coupled to the biomolecules has applications in the fields of e.g. molecular biology, biochemistry, pharmacology, and medical diagnostic technology.

### FIELD OF THE INVENTION

The present invention relates generally to methods and carriers for cross-linking or immobilising biomolecules such as polypeptides. In particular, the present invention relates to methods and carriers useful for coupling a polypeptide to a carrier via at least one disulphide bond. The carrier coupled to e.g. one or more polypeptide(s) has applications in the fields of e.g. molecular biology, biochemistry, pharmacology, and medical diagnostic technology.

### BACKGROUND OF THE INVENTION

Molecules can be immobilised on a carrier or solid surface either passively through hydrophobic or ionic interactions, or covalently by attachment to activated surface groups. In response to the enormous importance of immobilisation for solid phase chemistry and biological screening, the analytical uses of the technology have been widely explored. The technology has found broad application in many different areas of biotechnology, e.g. diagnostics, biosensors, affinity chromatography and immobilisation of molecules in ELISA assays. The value of immobilisation technology is demonstrated by the recent development of DNA micro arrays, where multiple oligonucleotide or cDNA samples are immobilised on a solid surface in a spatially addressable manner. These arrays have revolutionised genetic studies by facilitating the global analysis of gene expression in living organisms. Similar approaches have been developed for protein analysis, where as little as one picogram of protein need to be bound to each point on a micro array for subsequent analysis. The proteins bound to the micro arrays can then be assayed for functional or structural properties, facilitating screening on a scale, and with a speed previously unknown. The biomolecules bound to the solid surface may additionally be used to capture other unbound molecules present in mixture.

Development of this technology, with the goal of immobilising a biomolecule on a solid surface in a controlled manner, with minimal surface migration of the bound moiety and with full retention of its native structure and function, has been the subject of intensive investigation in recent years (Veilleux (1996) IVD Technology, March p. 26-31). The simplest type of protein immobilisation exploits the high inherent binding affinity of surfaces to proteins in general. For example proteins will physically adsorb to hydrophobic substrates via numerous weak contacts, comprising van der Waals, and hydrogen bonding interactions. The advantage of this method is that it avoids modification of the protein to be bound. On the other hand, proteins bound in this manner may be distributed unevenly over the solid support and/or inactivated since, for example, their clustering may lead to steric hindrance of the active site/binding region in any subsequent functional assay.

Alternative methods of immobilisation rely on the use of a few strong covalent bonds to bind the protein to the solid surface (Wilson D. S., Nock S., 2001, Current Opinion in Chemical Biology 6:81-85). Examples include immobilisation of biotinylated proteins onto streptavidin-coated supports, and immobilisation of His-tagged proteins, containing a poly-histidine sequence, to Ni²⁺-chelating supports. Other functional groups on the surface of proteins which can be used for attachment to an appropriate surface include reacting an amine with an aldehyde via a Schiff-base, cross-linking amine groups to an amine surface with gluteraldehyde to form peptide bonds, cross-linking carboxylic acid groups present on the protein and support surface with carbodiimide, cross-linking based on disulphide bridge formation between two thiol groups and the formation of a thiol-Au bond between a thiol group and a gold surface.

Amine coupling is a widely used method of immobilisation chemistry. N-hydroxysuccinimide esters are formed from a fraction of the carboxyl groups of the carboxymethyldextran matrix via reaction with N-hydroxysuccinimide (NHS) and N-ethyl-N′-(dimethylaminopropyl) carbodiimide hydrochloride (EDC) in water, which then react spontaneously with amine groups on a protein to form covalent bonds (Johnsson B., et. al., 1991, Anal Biochem 198:268-77). Following immobilisation, un-reacted N-hydroxysuccinimide esters on the support are deactivated with 1M ethanolamine hydrochloride to block areas devoid of bound proteins. The method is laborious since the reagents, used at each step of a chemical immobilisation method, usually need to be removed prior to initiating the next step.

Methods for the immobilisation of biomolecules via disulphide bridges are described by Veilleux J (1996) supra. Protein samples are treated with a mild reducing agent, such as dithiothreitol, 2-mercaptoethanol or tris(2-carboxyethyl)phosphine hydrochloride to reduce disulphide bonds between cysteine residues, which are then bound to a support surface coated with maleimide. Alternatively, primary amine groups on the protein can be modified with 2-iminothiolane hydrochloride (Traut's reagent) to introduce novel sulfhydryl groups, which are thereafter immobilised to the maleimide surface. Immobilisation of proteins on a gold substrate via a disulphide bridge is shown for the cupredoxin protein plastocyanin from Poplar (Andolfi, L. et al. 2002, Arch. Biochem. Biophys. 399: 81-88). Since this protein lacks a disulphide bridge, surface exposed residues Ile21 and Glu25 were both substituted with Cys. Disulphide bridge formation between the inserted cysteines was confirmed from the 3D crystal structure of the purified mutant plastocyanin. Mutant plastocyanin, expressed intracellularly in bacteria, is exposed to a reducing environment in the cytoplasm, such that the inserted cysteines are reduced, and can thus mediate the direct adsorption of the isolated protein onto a gold substrate. The thiol group binding properties of the protein are thus dependent on in vivo or in vitro chemical reduction of the cysteine residues on the surface of the protein.

An alternative approach to engineering thiol-group binding properties into a protein has been described for ribonuclease (RnaseA), which has four essential cysteines (Sweeney, R. Y. et al. 2000 Anal Biochem. 286: 312-314). In this case, a single cysteine residue was substituted for Ala19, located in a surface loop near the N-terminus of RNase A. The cysteine in the expressed RNase was protected as a mixed disulphide with 2-nitro-5-thiobenzoic acid. Following subsequent de-protection with an excess of dithiothreitol, the RNase was coupled to the iodoacetyl groups attached to a cross-linked agarose resin, without loss of enzymatic activity. Again, preparation of the protein for immobilisation requires its exposure to both protecting and de-protecting agents, which may negatively impact its native structure and/or function.

Methods are described in U.S. Pat. No. 6,350,368 for the coupling of FAD-dependent enzymes onto electrodes, whereby an apoenzyme is complexed with a functionalised FAD that is covalently bonded to a binding moiety, capable of chemical association (e.g. through a thiol group) or attachment on the surface of an electrode (e.g. gold). The immobilised FAD-enzyme complex is further functionalised with an electron mediator group and can be used in electrochemical systems for deforming analytes or optical signals.

Light-induced immobilisation techniques have also been explored, leading to the use of quinone compounds for photochemical linking to a carbon-containing support (e.g. as described in EP0820483). Activation occurs following irradiation with non-ionising electromagnetic radiation in the range from UV to visible light. Masks can be used to activate certain areas of the support for subsequent attachment of biomolecules. Following illumination the photochemically active compound, anthraquinone, will react as a free radical and form a stable ether bond with a polymer surface. Since anthraquinone is not found in native biomolecules, appropriate ligands have to be introduced into the biomolecule. In the case of proteins, this additional sample preparation step may require thermochemical coupling to the quinone and may not be site specific.

A further development of light-induced immobilisation technology is disclosed in U.S. Pat. No. 5,412,087 and U.S. Pat. No. 6,406,844, which describe a method for preparing a linker bound to a substrate. The terminal end of the linker molecule is provided with a reactive functional group protected with a photo-removable protective group, e.g. a nitro-aromatic compound. Following exposure to light, the protective group is lost and the linker can react with a monomer, such as an amino acid at its amino- or carboxy-terminus. The monomer, furthermore, may itself carry a similar photo-removable protective group, which can also be displaced by light during a subsequent reaction cycle. The method has particular application to solid phase synthesis, but does not facilitate orientated binding of proteins to a support. Bifunctional agents possessing thermochemical and photochemical functional substituents for immobilising an enzyme are disclosed in U.S. Pat. No. 3,959,078. Derivatives of arylazides are described which allow light mediated activation and covalent coupling of the azide group to an enzyme, and substituents which react thermochemically with a solid support. The orientation of the enzyme molecules immobilised by this procedure is not controlled.

A method for orientated, light-dependent, covalent immobilisation of proteins on a solid support, using the heterobifunctional wetting agent N-[m-[3-(trifluoromethyl)diazirin-3-yl]phenyl]-4-maleimidobutyramine, is described in WO 91/16425 and by Collioud A et al. (1993) in Bioconjugate Chem. 4: 528-536. The aryldiazirine function of this cross-linking reagent facilitates light-dependent, carbene-mediated, covalent binding to either inert supports or to biomolecules, such as proteins, carbohydrates and nucleic acids. The maleimide function of the cross-linker allows binding to a thiolated surface by thermochemical modification of cysteine thiols. Orientated binding of this cross-linking reagent to a protein can be attained by a thermochemical interaction between the maleimide function and an exposed thiol group on the protein surface; however this treatment may modify the structure and activity of the target protein. Light-induced covalent coupling of the cross-linking reagent to a protein via the carbene function, however, has the disadvantage that it does not provide for controlled orientation of the target protein.

WO 2004/065928 describes a method of cross-linking or immobilising proteins on a carrier wherein the carrier is modified with free thiol groups. The described method involves a method of coupling disulphide bridge containing proteins to a carrier by inducing the formation of thiol groups on a protein with irradiation, and coupling the protein to the carrier.

WO 94/01773 describes a method of photo-activation of proteins by UV to reduce sulphydryl groups for conjugation to radio-metals, chelating drugs, toxins etc. in vitro diagnosis, in vivo imaging and therapy.

Common for most of the described immobilisation methods is their use of one or more thermochemical/chemical steps, sometimes with hazardous chemicals, some of which are likely to have a deleterious effect on the structure and/or function of the bound protein. The available methods are often invasive, whereby foreign groups are introduced into a protein to act as functional groups, which cause protein denaturation, as well as lower its biological activity and substrate specificity.

Efficient and specific immobilisation procedures of biomolecules, in particular proteins and peptides, have wide interest in—for example—industries dealing with biosensors in particular and surface coatings in general.

Thus, there exists a need in the art of coupling and immobilisation of biomolecules for improved methods for coupling a broad range of biomolecules to different carriers, where the structural and functional properties of the coupled or immobilised component are preserved, the orientation of the biomolecule can be controlled, and the coupling can be spatially controlled to specific activated regions.

### SUMMARY OF INVENTION

Methods and carriers and use thereof are provided for immobilisation or coupling of polypeptides to carriers. Thus, the invention provides a method of coupling a polypeptide to a carrier via at least one disulphide bond, said carrier comprising a support, which support is attached to at least one disulphide-containing linker capable of being activated by irradiation to contain reactive thiol groups, comprising the following steps of:
a. incubating a polypeptide containing at least one reactive thiol group with the carrier,
b. irradiating the carrier to create reactive thiol groups,
or
a. irradiating a polypeptide containing at least one disulphide bridge to create reactive thiol groups in the polypeptide by disulphide bridge disruption,
b. irradiating the carrier to create reactive thiol groups, and
c. incubating the irradiated polypeptide with the irradiated carrier, wherein step a and step b can be simultaneous or sequential in any order.

In another aspect, the invention provides the use of the method according to the invention for the production of a polypeptide-based surface coating for use in the production of polypeptide-based biosensors, polypeptide-based micro arrays and food packing materials with polypeptide-based surface coatings, and the uses thereof.

In a further aspect, the invention provides a carrier comprising a support, which is attached to at least one disulphide-containing linker capable of being activated by irradiation to contain reactive thiol groups and after activation being coupled to a polypeptide, and the uses thereof.

In another aspect, the invention provides a carrier coupled to one or more polypeptides obtainable by the method according to the invention, and the uses thereof.

In a further aspect, the invention provides a method of delivering a drug or prodrug to a patient comprising the following steps of:
a. providing a carrier coupled to one or more polypeptides
b. administering the carrier-coupled polypeptide to a patient
c. irradiating the carrier-coupled polypeptide to create a thiol group in the molecule by disulphide bridge disruption and thereby releasing the polypeptide from the carrier.

### BRIEF DESCRIPTION OF THE DRAWINGS

FIG. 1 is an embodiment according to the invention and shows a carrier comprising a support attached to a cyclic peptide linker CXWXC (cf. SEQ ID NO. 1) wherein C is cysteine groups joined via a disulphide bridge which disulphide bridge is disrupted when irradiated to create reactive thiol groups, W is tryptophan and X is any amino acid which does not comprise a reactive thiol group, and Y is part of the support capable of attachment to the peptide linker. In this embodiment the polypeptide contains a disulphide bridge which is activated to contain reactive thiol groups when the protein and the peptide linker are irradiated.

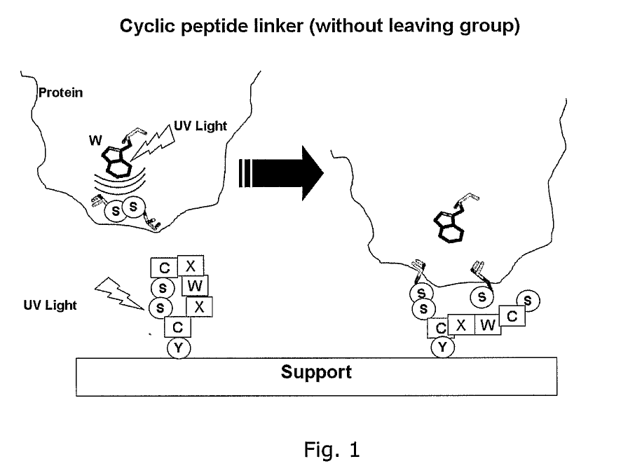

FIG. 2 is an embodiment according to the invention and shows a carrier comprising a support of aldehyd derivatised silicon attached via a shiffs-bond to the nitrogen atom (N) in lysine of a cyclic peptide linker KXXXXWXCXXXC (cf. SEQ ID NO. 2), wherein C is cysteine groups joined via a disulphide bridge which disulphide bridge is disrupted when irradiated to create reactive thiol groups, W is tryptophan, K is lysine and X is any amino acid which does not comprise a reactive thiol group. In this embodiment the polypeptide contains a disulphide bridge which is activated to contain reactive thiol groups when the protein and the peptide linker are irradiated.

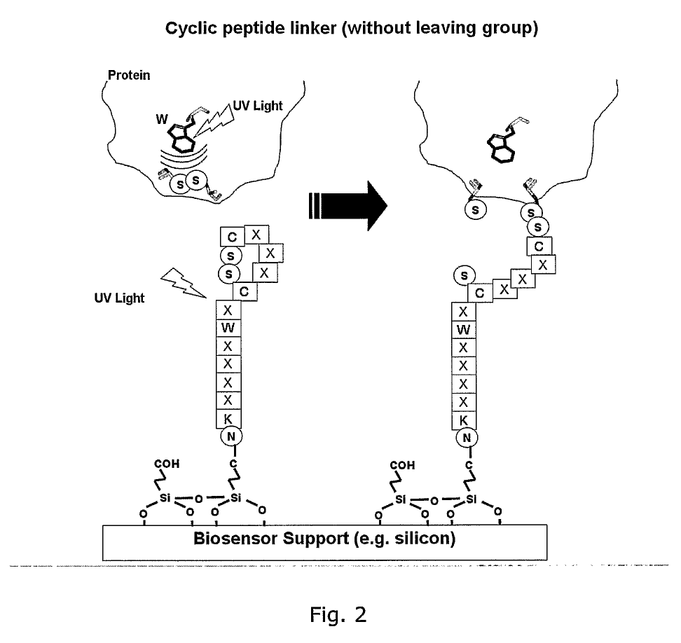

FIG. 3 is an embodiment according to the invention and shows a carrier comprising a support of aldehyd derivatised silicon attached via a shiffs-bond to the nitrogen atom (N) in lysine of a non-cyclic peptide linker KXXXXWXC-S-S-CXX (cf. SEQ ID NO. 3 and 4), wherein C is cysteine groups joined via a disulphide bridge, W is tryptophan, K is lysine and X is any amino acid which does not comprise a reactive thiol group. In this embodiment, part of the linker is a leaving group. In this embodiment the polypeptide contains a disulphide bridge which is activated to contain reactive thiol groups when the protein and the peptide linker are irradiated.

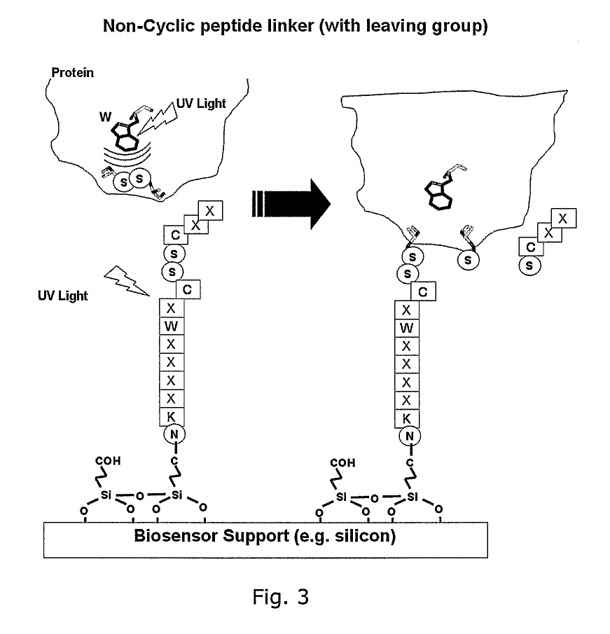

FIG. 4 is an embodiment according to the invention and shows a carrier comprising a support of aldehyd derivatised silicon attached via a shiffs-bond to the nitrogen atom (N) in cysteine (C) of a cyclic peptide linker CXWXC (cf. SEQ ID NO. 1), wherein C is cysteine groups joined via a disulphide bridge, W is tryptophan, and X is any amino acid which does not comprise a reactive thiol group. In this embodiment the polypeptide contains a disulphide bridge which is activated to contain reactive thiol groups when the protein and the peptide linker are irradiated.

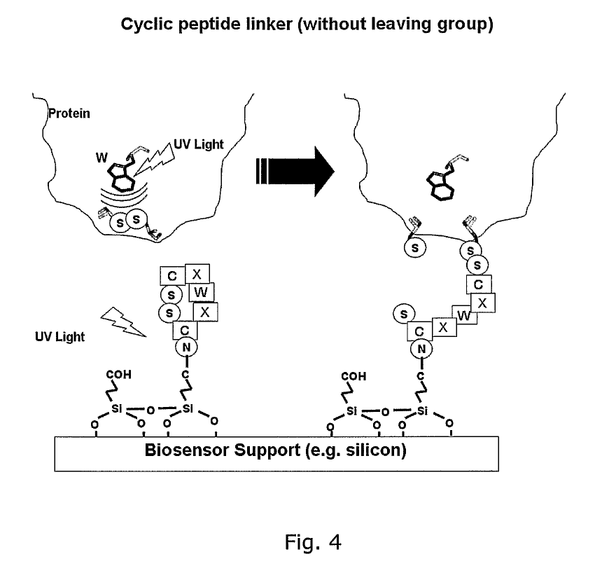

FIG. 5 is an embodiment according to the invention and shows a carrier comprising a support of aldehyd derivatised silicon attached via a shiffs-bond to the nitrogen atom (N) in cysteine of a non-cyclic peptide linker C-S-S-CXW (cf. SEQ ID NO. 5), wherein C is cysteine groups joined via a disulphide bridge, W is tryptophan, and X is any amino acid which does not comprise a reactive thiol group. In this embodiment, part of the linker is a leaving group. In this embodiment the polypeptide contains a disulphide bridge which is activated to contain reactive thiol groups when the protein and the peptide linker are irradiated.

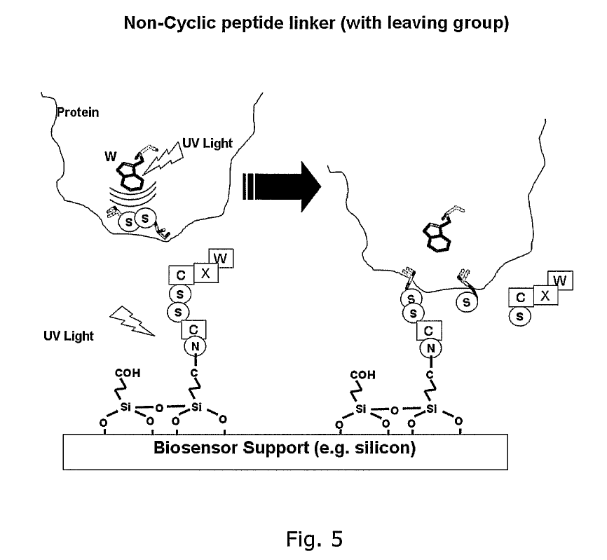

FIG. 6 is an embodiment according to the invention and shows a carrier comprising a support of aldehyd derivatised silicon attached via a shiffs-bond to the nitrogen atom (N) in lysine of a non-cyclic peptide linker KXXXXWXCCX (cf. SEQ ID NO. 6), wherein C is cysteine groups joined via a disulphide bridge and via a peptide bond, W is tryptophan, K is lysine and X is any amino acid which does not comprise a reactive thiol group. In this embodiment the polypeptide contains a disulphide bridge which is activated to contain reactive thiol groups when the protein and the peptide linker are irradiated.

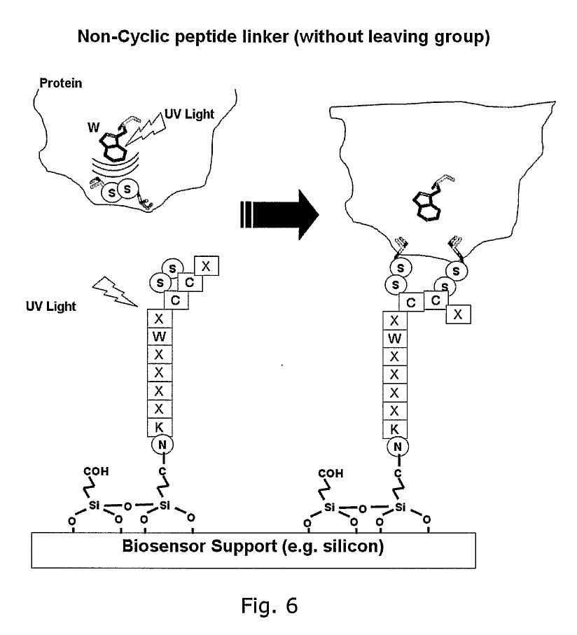

FIG. 7 is an embodiment according to the invention and shows a carrier comprising a support of aldehyd derivatised silicon attached via a shills-bond to the nitrogen atom (N) in cysteine of a non-cyclic peptide linker CCXW (cf. SEQ ID NO. 7), wherein C is cysteine groups joined via a disulphide bridge and a peptide bond, W is tryptophan, and X is any amino acid which does not comprise a reactive thiol group. In this embodiment the polypeptide contains a disulphide bridge which is activated to contain reactive thiol groups when the protein and the peptide linker are irradiated.

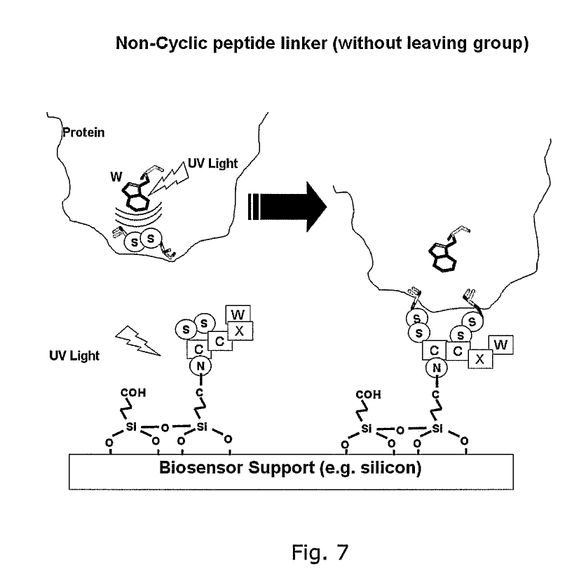

### DETAILED DESCRIPTION OF THE INVENTION

The present invention provides a method for coupling a polypeptide to a carrier via one or more stable bond(s) (covalent disulphide bond(s)). In a preferred embodiment of the invention, the native structural and functional properties of the coupled polypeptide, such as a protein, can be preserved and the use of one or more chemical and/or thermal steps can be avoided. This contrasts with traditional coupling methods for protein immobilisation, which typically involve several chemical and/or themal reactions, which can be costly, time-consuming as well as deleterious to the structure/function of the bound protein. Furthermore, the orientation of the protein, coupled according to the method of the present invention, can in one aspect of the invention be controlled, such that the functional properties e.g. enzymatic are preserved. In comparison, the majority of known protein coupling methods lead to a random orientation of the proteins immobilised on a carrier, with the significant risk of lower biological activity and thereby e.g. raised detection limits.

The present invention describes a method where the carrier is modified in such a way that it does require pre-treatment with irradiation before it can react with polypeptides to form a covalent coupling. The method according to the invention involves the use of a carrier comprising a support to which is attached disulphide-containing linker(s) which is convertible by irradiation to contain fully reactive thiol(s) capable of immobilising a desired polypeptide, said polypeptide containing at lest one reactive thiol group or at least one disulphide bridge capable of being activated by irradiation to reactive thiol groups. This makes it possible to selectively irradiate pre-determined regions of the carrier to convert the disulphide-containing linker to contain reactive thiol(s) and thereby obtain immobilisation of the polypeptide on the activated regions of the carrier. This spatially addressability is a major advantage afforded by the method of the invention.

Accordingly, the present invention relates to a method of coupling a polypeptide to a carrier via at least one disulphide bond, said carrier comprising a support, which support is attached to at least one disulphide-containing linker capable of being activated by irradiation to contain reactive thiol groups, comprising the following steps of:
a. incubating a polypeptide containing at least one reactive thiol group with the carrier,
b. irradiating the carrier to create reactive thiol groups,
   or
a. irradiating a polypeptide containing at least one disulphide bridge to create reactive thiol groups in the polypeptide by disulphide bridge disruption,
b. irradiating the carrier to create reactive thiol groups, and
c. incubating the irradiated polypeptide with the irradiated carrier,
   wherein step a and step b can be simultaneous or sequential in any order.

As used herein, the terms “UV light” or “irradiation” or UV illumination” or “UV irradiation” are a range of wavelengths or a single wavelength of UV light.

As used herein, the term “carrier” describes the linker attached to the support.

As used herein, “reactive thiol group” relates to a thiol group which are capable of covalent coupling to another thiol group creating a disulphide bond.

The disulphide-containing linker used in the method according to the invention is designed so it is activated by irradiation e.g. UV-beam illumination and thereby making spatially controlled immobilisation possible. This is an advantage of the present method compared to known methods for spot-size immobilisation using UV-beam immobilisation where the carrier or surface is capable of binding without activation. When the polypeptide to be immobilised is in a solution, known spot-size immobilisation methods may allow polypeptide that were activated above the surface to diffuse to an area outside the UV-beam and be immobilised. Using the method according to the present invention, this problem is solved since immobilisation now requires activation of the carrier. Activated polypeptide that diffuses outside the immobilisation area will not immobilise because the surface outside e.g. the UV-beam area is not activated using the present method. In one aspect of the invention, the immobilisation is spatially controlled.

In one aspect of the invention, the polypeptide contains a disulphide bridge and the disulphide-containing linker and the polypeptide are activated by irradiation to create reactive thiol groups in the same step or sequentially.

The invention thus relates to a method of coupling a polypeptide to a carrier via at least one disulphide bond, said carrier comprising a support which support is attached to at least one disulphide-containing linker capable of being activated by irradiation to contain reactive thiol groups, which method comprises the following steps of:
a. irradiating a polypeptide containing at least one disulphide bridge to create reactive thiol groups in the polypeptide by disulphide bridge disruption,
b. irradiating the carrier to create reactive thiol groups, and
c. incubating the irradiated polypeptide with the irradiated carrier,
   wherein step a and step b can be simultaneous or sequential in any order.

In one aspect of the invention, a method of coupling a polypeptide to a carrier via at least one disulphide bond, said carrier comprising a support which support is attached to at least one disulphide-containing linker capable of being activated by irradiation to contain reactive thiol groups, which method comprises the following steps of:
a. incubating a polypeptide containing at least one reactive thiol group with the carrier,
b. irradiating the carrier to create reactive thiol groups,
   wherein step a and step b can be simultaneous or sequential in any order, is provided.

In one aspect of the invention, step a and step b is performed simultaneous. In another aspect of the invention, first step a is performed and then step b. In another aspect of the invention, first step b is performed and then step a.

It is, thus, also possible according to the invention to immobilise polypeptides already containing at least one reactive thiol group. According to this aspect of the invention, only activation of the carrier is necessary. The polypeptide may contain such reactive thiol group(s) in their native form or they may have been formed after chemical treatment.

As used in the present invention, a disulphide-containing linker is a molecule which is capable of being attached to the support and to be activated by irradiation to contain reactive thiol group(s) (—SH group(s)).

When activated the thiol group should preferably be pointing upwards and away from the support to be readily available for coupling with the polypeptide.

A disulphide-containing linker may include but is not limited to a linker comprised solely or partly by amino acids. In one aspect of the invention, the linker is comprised solely by amino acids.

The term “amino acid” comprises both natural amino acids such as Ala (alanine), Cys (cysteine), Asp (aspartic acid), Glu (glutamic acid), Phe (phenylalanine), Gly (glycine), His (histidine), Ile (isoleucine), Lys (lysine), Leu (leucine), Met (methionine), Asn (asparagines), Pro (proline), Gln (glutamine), Arg (arginine), Ser (serine), Thr (threonine), Val (valine), Trp (tryptophan), Tyr (tyrosine) and unnatural or modified amino acids such as Aad (2-aminoadipic acid), bAad (3-Aminoadipic acid), bAla (beta-alanine, beta-aminopropionic acid), Abu (2-aminobutyric acid), 4Abu (4-aminobutyric acid, piperidinic acid), Acp (6-aminocaproic acid), Ahe (2-aminoheptanoic acid), Aib (2-aminoisobutyric acid), bAib (3-aminoisobutyric acid), Apm (2-aminopimelic acid), Dbu (2,4 diaminobutyric acid), Des (desmosine), Dpm (2,2′-diaminopimelic acid), Dpr (2,3-diaminopropionic acid), EtGly (N-ethylglycine), EtAsn (N-ethylasparagine), Hyl (hydroxylysine), aHyl (allo-hydroxylysine), 3Hyp (3-hydroxyproline), 4Hyp (4-hydroxyproline), Ide (isodesmosine), aIle (allo-isoleucine), MeGly (N-methylglycine, sarcosine), MeIle (N-Methylisoleucine), MeLys (6-N-methyllysine), MeVal (N-methylvaline), Nva (norvaline), Nle (norleucine) and Orn (ornithine). The amino acids are preferably natural amino acids. In the formulas, the IUPAC amino acid letter code is used.

A disulphide-containing linker as used in the present context may include other molecules than amino acids and may be comprised by one or more peptide groups and one or more groups of organic or non-organic materials, e.g. containing a peptide group and one or more carbohydrate groups, including small sugar molecules, oligosaccharides, large carbohydrate-based polymers. Inorganic part(s) of the linker may include e.g. metallic groups based on gold, silver, aluminium, silicon, and/or non-metallic groups based e.g. on ceramic.

The disulphide-containing linker is in one aspect of the invention a peptide linker comprising at least one amino acid. In a further aspect, the peptide linker comprises at least one aromatic amino acid.

When the disulphide-containing linker is activated by irradiation to contain reactive thiol groups, part of the linker may be set free (a leaving group) as a by-product. The by-product (or leaving group) will usually be washed away from the surface if they interfere with subsequent reactions. The free thiol group still part of the linker can participate in the formation of a new disulphide bond to a free thiol in the polypeptide. It is preferred that the disulphide-containing linker is designed so as to not leave any by-product.

In a further aspect of the invention, the peptide linker has formula I

-L-D (formula I),

wherein L is attached to the support and comprises at least one amino acid which does not contain a reactive thiol and which is different from an amino acid which is capable of being activated by irradiation to contain at least one reactive thiol group and D is a non-cyclic sequence of amino acids or a cyclic sequence of amino acids, which non-cyclic or cyclic sequence comprises at least two cysteines (C) covalently joined by a disulphide bridge and wherein one of the cysteines (C) is bound to L. In one aspect of the invention the two cysteines (C) are covalently joined by a disulphide bridge (-C-S-S-C-). In another aspect they are covalently joined by a disulphide bridge and also by a peptide bond (-C-C-). It is preferred that the peptide linker comprises at least one aromatic amino acid which may be part of either -D or -L.

According to one aspect of the invention, L comprises 1-30 amino acids, preferably 3-20 amino acids and most preferred 5-10 amino acids. According to a further aspect of the invention, D comprises 2-30 amino acids, preferably 3-20 amino acids and most preferably 5-10 amino acids.

According to still another preferred aspect of the invention, L comprises one or more aromatic amino acids, such as e.g. tryptophan. In a further aspect of the invention, L comprises an aromatic amino acid separated from the cysteine (C) in D bound to L by at least one amino acid.

In a further aspect of the invention, D is a cyclic sequence of amino acids.

In yet a further aspect of the invention, the C-terminus is amidated in order to prevent reactions between the C-terminal carboxyl group and the polypeptide.

According to one aspect of the invention, D has the following sequence C(X)ₙC, wherein X independently is any amino acid which does not comprise a reactive thiol group, n is from 1 to 10, preferably from 2 to 8 and more preferably from 3 to 6, and the two cysteines (C) are covalently joined by a disulphide bridge. In another aspect of the invention, D has the following sequence CC(X₁)ₙ₁, wherein X₁ independently is any amino acid which does not comprise a reactive thiol group, n1 is from 0 to 10, preferably from 2 to 8 and more preferably from 3 to 6, and the two cysteines (C) are covalently joined by a disulphide bridge and a peptide bond. In another aspect of the invention D has the following sequence C-S-S-C(X₁)ₙ₁, wherein X₁ independently is any amino acid which does not comprise a reactive thiol group, n1 is from 0 to 10, preferably from 2 to 8 and more preferably from 3 to 6, and the two cysteines (C) are covalently joined by a disulphide bridge.

In a further aspect of the invention, L has the following sequence (X₃)ₙ₃W(X₄)ₙ₄, wherein X₃ and X₄ independently are any amino acid which does not comprise a reactive thiol group, W is tryptophan and n3 and n4 each independently are from 1 to 5. In one aspect of the invention, n3 is 5. In another aspect of the invention, n3 is 3. In a further aspect of the invention, n4 is 1.

In one aspect of the invention, the peptide linker is CXWXC (cf. SEQ ID NO. 1) wherein C is cysteine groups joined via a disulphide bridge, W is tryptophan and X is any amino acid which does not comprise a reactive thiol group.

In another aspect of the invention, the peptide linker is KXXXXWXCXXXC (cf. SEQ ID NO. 2), wherein C is cysteine groups joined via a disulphide bridge, W is tryptophan, K is lysine and X is any amino acid which does not comprise a reactive thiol group.

In another aspect of the invention, the peptide linker is KXXXXWXC-S-S-CXX (cf. SEQ ID NO. 3 and 4), wherein C is cysteine groups joined via a disulphide bridge, W is tryptophan, K is lysine and X is any amino acid which does not comprise a reactive thiol group. In this embodiment, part of the linker is a leaving group.

In another aspect of the invention, the peptide linker is C-S-S-CXW (cf. SEQ ID NO. 5), wherein C is cysteine groups joined via a disulphide bridge, W is tryptophan, and X is any amino acid which does not comprise a reactive thiol group. In this embodiment, part of the linker is a leaving group.

In another aspect of the invention, the peptide linker is KXXXXWXCCX (cf. SEQ ID NO. 6), wherein C is cysteine groups joined via a disulphide bridge and via a peptide bond, W is tryptophan, K is lysine and X is any amino acid which does not comprise a reactive thiol group.

In another aspect of the invention, the peptide linker is CCXW (cf. SEQ ID NO. 7), wherein C is cysteine groups joined via a disulphide bridge and a peptide bond, W is tryptophan, and X is any amino acid which does not comprise a reactive thiol group.

In yet a further aspect of the invention, L-D has the following sequence K(X₅)ₙ₅WX₆CGGGC, wherein X₅ and X₆ independently are any amino acid which does not comprise a reactive thiol group, W is tryptophan, K is lysine, n5 is 3, G is glycine and the two cysteine molecules (C) are covalently joined by a disulphide bridge. In another aspect of the invention, L-D has the following sequence K(X₅)ₙ₅WX₆CGGGC, wherein X₅ and X₆ independently are any amino acid which does not comprise a reactive thiol group, W is tryptophan, K is lysine, n5 is 4, G is glycine and the two cysteine molecules (C) are covalently joined by a disulphide bridge. In yet a further aspect of the invention, L-D has the following sequence KAMHAWGCGGGC-NH2 (cf. SEQ ID NO. 9), wherein CGGGC (cf. SEQ ID NO. 8) is cyclic and the two cysteine molecules (C) are covalently joined by a disulphide bridge, K is lysine, A is alanine, M is methionine, H is histidine, W is tryptophan, and G is glycine.

In another aspect of the invention, L-D has the following formula KAMHAWGC-S-S-CX₇X₈-NH2 (cf. SEQ ID NO. 10 and 12), wherein X₇ and X₈ independently are any amino acid which does not comprise a reactive thiol group, the two cysteine molecules (C) are covalently joined by a disulphide bridge, K is lysine, A is alanine, M is methionine, H is histidine, W is tryptophan, and G is glycine, preferably the following formula KAMHAWGC-S-S-CGG-NH2 (cf. SEQ ID NO 10 and 11), wherein the two cysteine molecules are covalently joined by a disulphide bridge, K is lysine, A is alanine, M is methionine, H is histidine, W is tryptophan, and G is glycine.

In a further aspect of the invention in any of the above sequences, any of X, X₁, X₂, X₃, X₄, X₅, X₆, X₇, and X₈ are independently any amino acid which does not comprise a reactive thiol group. In any of the above sequences, any of X₁, X₂, X₃, X₄, X₅, X₆, X₇, and X₈ are in a further aspect of the invention, independently any amino acid except cysteine. In a further aspect of the invention, the amino acid is selected from the group consisting of basic amino acids such as Lys, Arg or His, acidic and amidic amino acids such as Asp, Glu, Asn or Gln, amino acids with non-charged side chains such as Gly, Ala, Val, Leu, Ile, Pro, Ser, Thr, Met, Phe, Tyr or Trp. In a further aspect of the invention, the amino acid is selected from the group consisting of Gly, Ala, Val, Leu or Ile. In another aspect of the invention, the amino acid is selected from the group consisting of Gly, Ala, Val or Leu, and in a further aspect the amino acid is selected from the group consisting of Gly, Ala or Val and in still a further aspect the amino acid is selected from the group consisting of Gly or Ala such as Gly.

In one aspect according to the invention, -D (formula II) is as described above and is directly attached to the support. In one aspect of the invention, -D comprises preferably an aromatic amino acid such as tryptophan, tyrosine and phenylalanine. In a further aspect of the invention, the aromatic amino acid is tryptophan.

Many methods are available for the skilled person for making the disulphide-containing linker used in the present invention such as e.g. described in Kimmerlin T. Seebach D. (2004). ‘100 years of peptide synthesis’: ligation methods for peptide and protein synthesis with applications to b-peptide assemblies', J Pept Res, Vol 65, Issue 2, pp. 229-260, McKay, F. C. & Albertson, N. F. (1957) New amine-masking groups for peptide synthesis. J. Am. Chem. Soc. 79, 4686-4690 and Carpino, L. A. (1957) Oxidative reactions of hydrazines. IV. Elimination of nitrogen from 1,1-disubstituted-2-arensulfonhydrazides. J. Am. Chem. Soc. 79, 4427-4430, incorporated herein by reference.

The term “polypeptide” is in the present context intended to mean molecules comprising polyamino acids covalently linked via peptide bonds, and the term encompasses both short peptides of from 2 to 10 amino acid residues, and oligopeptides of from 10 to 20 amino acid residues, and polypeptides of more than 20 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups. Thus, the term includes enzymes, antibodies, antigens, transcription factors, immunoglobulin, binding proteins e.g. DNA binding proteins, or protein domains or fragments of proteins or any other amino acid based material. The term “polyamino acid” denotes a molecule constituted by at least 3 covalently linked amino acid residues.

In one aspect of the invention, the polypeptide comprises one or more aromatic amino acid(s) which is/are selected from the group consisting of tryptophan, tyrosine and phenylalanine.

The polypeptide is in one aspect of the invention selected from the group consisting of an enzyme such as selected from the group consisting of cutinase, chymosin, glucose oxidase, lipase, lysozyme, alkaline phosphatase and plasminogen, a transcription factor, a protein domain, a binding protein, an antigen and an immunoglobulin, such as a F(ab) fragment.

In the aspect of the invention where the polypeptide contains a disulphide bridge, an inherent property of e.g. proteins, concerning irradiation-induced structural changes, thought to retard their photo-degradation, is exploited. When proteins are exposed to UV irradiation, some disulphide bridges are disrupted to form activated thiols. Although disulphide bridges are commonly found in the structural core and near/on the surface of folded proteins, those located in close proximity to aromatic amino acids are the most susceptible to UV-induced disruption. During UV exposure of proteins, energy absorbed by side chains of aromatic amino acid residues is transferred to spatial neighbouring disulphide bridges, which function as quenchers (Neves-Petersen M T., et al., 2002, Protein Science 11: 588-600). However, the flow of energy transferred to disulphide bridges and the likely formation of intermediate chemical species such as radicals/ions formed upon light excitation of the sample ultimately serves to trigger their disruption. The presence of a disulphide bridge with tryptophan as a close spatial neighbour in a protein occurs frequently in nature, indicating that photo-induced disulphide bridge disruption is a widespread phenomenon (Petersen M T N., et al., 1999, Protein Engineering 12: 535-548; Neves-Petersen M T et al., 2002, Protein Science 11: 588-600; Vanhooren A et al. 2002, Biochemistry 10; 41(36):11035-11043).”

As used herein, the term “spatial neighbour” relates to the physical distance between two chemical groups within a composition, such that groups lying in three-dimensional close proximity are considered to be spatial neighbours. A disulphide bridge in e.g. a protein which is a spatial neighbour to an aromatic residue may function as a quencher if the aromatic amino acid absorbs excitation energy following irradiation. The physical distance between half cystines of a disulphide bridge, which are spatial neighbours to one or more aromatic residues such as tryptophan residues and may act as quenchers, can be, but is not limited, to a range of 1 to 10 Å. In one aspect of the invention, the polypeptide, such as a protein, comprises a disulphide bridge, which is spatial neighbours to one or more aromatic residues, in said polypeptide.

Cutinase, from the fungus Fusarium solani pisi, is one of several proteins that may be coupled on a carrier. Cutinase is a lipolytic enzyme capable of degrading cutin, an insoluble lipid-polyester matrix found on the surface of plant leaves. Cutinase is an industrially important enzyme, and it is proposed to incorporate in detergents for the removal of fats. It has two disulphide bridges; one near the active site, and one distal at the opposite pole of the protein, in close proximity to the single tryptophan residue of the protein. Chemical reduction of the disulphide bridge, located near the active site, renders the enzyme inactive (Soliday C L, et. al., 1983, Biochem Biophys Res Commun 114:1017-22).

In its native conformation, the single tryptophan residue of F. solani pisi cutinase is highly quenched due to the presence of the adjacent disulphide bridge. Following prolonged selective irradiation of cutinase at 295 nm, the fluorescence quantum yield of the single Trp residue increases simultaneously with the disruption of the neighbouring disulphide bridge. The increased quantum yield of the Trp residue in cutinase reflects the loss of the disulphide bridge and its ability to quench the Trp residue in an excited-state.

Disulphide bridges are known to be excellent quenchers of excited-state aromatic residues. Any aromatic residue, which is in close spatial proximity, can cause photo-induced disruption of a neighbouring disulphide bridge. Hence, the three aromatic amino acids, tryptophan, tyrosine and phenylalanine found in proteins, are all potential mediators of light-induced disulphide bridge disruption. While irradiation with light of a range of wavelengths extending from 240 nm to 300 nm will excite all aromatic residues, the individual aromatic residues have differing absorption maxima (Table 1; data obtained at neutral pH).

TABLE 1

| In water | Absorption Max. | Emission Max. |
|---|---|---|
| Phe | 254 nm | 282 nm |
| Tyr | 275 nm | 303 nm |
| Trp | 280 nm | 250 nm |

Since the excitation spectrum of the aromatic amino acid residues is only partially overlapping, protein irradiation at a single r narrow wavelength range will excite the individual residues to different degrees. Irradiation at 295 nm can be used to selectively excite tryptophan residues in a protein. Irradiation at 280 nm will excite both tyrosine and tryptophan residues, which can both then cause photo-induced disulphide bridge disruption. Where irradiation is performed by multiple-photon excitation, for example when two-photon excitation is carried out, the sample is irradiated with photons (light) with half the energy (twice the wavelength) of the photons used in a single-photon experiment. For example, electronic excitation of tryptophan can both be achieved with ultraviolet light at 295 nm, or with two-photon excitation at a wavelength of approximately 690 nm. Furthermore, excited tyrosine residues can cause the excitation of neighbouring tryptophan residues by a mechanism called fluorescence resonance energy transfer, which in turn can cause disulphide bridge disruption.

The polypeptide may comprise one or more disulphide bridges or reactive thiols and the disulphide linker may after irradiation thus be coupled to the polypeptide with either one or more disulphide bonds. It is preferred that the polypeptide only contains one disulphide bridge which is capable of being activated to reactive thiol groups by irradiation.

In a preferred aspect, the polypeptide comprises one disulphide bridge, and in a further aspect the polypeptide and the carrier are irradiated simultaneously to create reactive thiol groups.

In another aspect of the invention, the free thiols in said polypeptide are formed after chemical treatment e.g. using a disulphide reducing agent such as DTT.

In an aspect of the invention, the irradiation is preferably performed with a light source of a wavelength of between about 250 nm and about 320 nm, more preferred between about 275 nm and about 300 nm, or IR/visible light for multi-photon excitation. In another aspect of the invention the irradiation step comprises light of a wavelength that specifically excites one or more aromatic amino acids, or other molecular system that may mimic aromatic amino acids, preferably said irradiation step comprises light of a wavelength that excites one specific aromatic amino acid such as e.g. the wavelength of approximately e.g. 295 nm, 275 nm or 254 nm that excites respectively tryptophan, tyrosine or phenylalanine, most preferably the wavelength about 295 nm that excites tryptophan or the wavelength about 275 nm that excites tyrosin, or multi-photon excitation, for example 2-photon excitation of between e.g. 500 nm and 640 nm or 3-photon excitation of between 750 nm and 960 nm.

In one aspect of the invention, the irradiation is performed by multi-photon excitation, preferably by two-photon excitation. In another aspect of the invention, the irradiation comprises light with a wavelength of about 250-320 nm. In a further aspect of the invention, the irradiation comprises light with a wavelength of about 275-300 nm. In yet a further aspect of the invention, the irradiation comprises light with a wavelength of about 295 nm, 275 nm or 254 nm.

In another aspect of the invention, wherein the polypeptide comprises one or more aromatic amino acids such as tryptophan, the irradiation is performed at a wavelength of about 295 nm.

In another aspect of the invention, wherein the polypeptide comprises one or more aromatic amino acids such as tyrosin, the irradiation is performed at a wavelength of about 275 nm.

In one aspect of the invention, the polypeptide is irradiated in the presence of a free aromatic amino acid, such as Trp, Tyr and Phe.

It will be apparent to those skilled in the art that the disruption of disulphide bonds in a given protein at a selected wavelength can be predicted from the location and amino acid neighbours of each disulphide bridge in the 3D structure of the protein. Disulphide bridges placed in the spatial vicinity of aromatic amino acid residues are likely to be the most labile to UV light. The 3D structures of a subset of proteins containing the spatial triad Trp Cys-Cys, in close spatial proximity, have been examined in order to identify which amino acids are located in immediate vicinity of the tryptophan residues of the triad (WO 2004/065928). This analysis has identified those proteins having a similar amino acid neighbourhood composition around the triad to that of cutinase, which can be used to predict which proteins will have the disulphide bond of the triad broken upon UV illumination.

One such protein is goat alfa-lactalbumin, where the amino acid residues located within an 8 Å sphere around the tryptophan residue of the triads of goat alfa-lactalbumin are very similar to that of cutinase. Furthermore, it has recently been shown that upon UV excitation of goat-lactalbumin, those disulphide bridges lying adjacent to tryptophan residues, are disrupted and free thiol groups are formed (Vanhooren A et al. 2002, supra). It is also possible to predict the UV stability of putative disulphide bridges in a given protein, solely on the basis of its primary structure, i.e., its amino acid sequence, provided that the 3D structure of homologous proteins is known and can be used for homology modelling of the 3D structure of the given protein. Among other proteins, the list of proteins having an aromatic residue in close proximity to a disulphide bridge that can be used for immobilisation includes: Immunoglobulin Fab fragments, Major Histocompatibility Complex (MHC class I and class II), Alkaline Phosphatase, Plasminogen, Lysozyme, Trypsin, Pepsin, Cutinase.

It will also be apparent that known methods of recombinant DNA technology can be used to introduce amino acid substitutions into a protein sequence to create additional photo-disruptable disulphide bridges. Such substitutions may introduce a tryptophan residue or other aromatic amino acid residue in a protein as close spatial neighbour of endogenous or recombinantly engineered disulphide bridge, or alternatively a disulphide bridge may be introduced in close spatial proximity to an endogenous aromatic residue. Alternatively both aromatic and disulphide bridge may be introduced in close spatial proximity to each other. Irradiation with 295 nm light is preferable since it permits the selective excitation of tryptophan residues in a protein, which in turn may lead to the disruption of a single or a limited number of disulphide bonds.

A variety of light sources suitable for the irradiation of proteins at a range of wavelengths, for the photo-induction of disulphide bond disruption include, but are not limited to, a 75-W Xenon arc lamp from a research grade spectrometer such as a RTC PTI spectrometer, a deuterium lamp, a high pressure mercury lamp. Irradiation at a single wavelength can be obtained by coupling the light source to a monochromator. A source of single and multiple photon excitation includes a high peak-power pulsed or CW laser.

New thiol groups are formed in proteins following light-induced disulphide bond disruption. Their appearance de novo can be measured by a 5,5′-dithiobis-(2-nitrobenzoic acid) [DTNB] based assay. In the case of cutinase, irradiated at 295 nm, the formation of one thiol group per illuminated protein was detected on average. There are no free thiols in native cutinase, and disruption of the disulphide adjacent to the single tryptophan residue only yields one solvent accessible thiol that can be detected by this method. Light-induced thiol groups formed on proteins, which are accessible, will bind to any thiol binding ligand or free thiol group on a carrier.

In an aspect, the method according to the invention further comprises the steps of:

verifying the presence of one or more disulphide bridges in said polypeptide,
identifying one or more aromatic amino acid residues in close spatial proximity to said one or more disulphide bridges,
selecting a wavelength which specifically excites one or more of said aromatic amino acid residues, thereby disrupting one or more of said disulphide bonds.

The support to which the disulphide-containing linker is attached may be any biological, non-biological, organic, inorganic or a combination of any of these materials existing as particles, strands, precipitates, gels, sheets, tubing, spheres, containers, capillaries, pads, slices, films, plates, slides, etc. It can have any convenient shape.

As used herein, the term “support” can be a soluble, semi-soluble or insoluble material to which the linker is capable of being attached. Examples of an insoluble support is electronic chips, slides, wafers, particles, resins, wells, tubes or membranes which include but are not limited to any material comprising polymers such as Topaz, polystyrene, polyethylene, polyester, polyethermides, polypropylene, polycarbonate, polysulfone, polymethylmethacrylate [PMMA], poly(vinylidene flouride) [PVDF], siliciumoxide containing materials such as silicon; diamond; glass e.g. quartz and silica; silicon e.g. silicon wafers; metals such as gold, silver and aluminium; membranes e.g. nylon membranes, nitrocellulose filters; porous materials such as gels, agarose or cellulose; ceramics etc. which furthermore include all forms of derivatisation of the support which facilitate attachment or binding of the peptide linker to the support. Examples of a soluble support are a soluble compound or polymer such as hydrocarbons or another biomolecule, for example another polypeptide. In one aspect of the invention, the support is a polypeptide or another biomolecule such as DNA or synthetic biomolecules such as aptamers.

Methods for attaching the linker to the support will be apparent to those skilled in the art and comprise e.g. binding an amine in the peptide linker with an aldehyde via a Schiff-base, cross-linking amine groups in the peptide linker to an amine surface with gluteraldehyde to form peptide bonds, cross-linking carboxylic acid groups present in the peptide linker and support surface with carbodiimide, cross-linking based on disulphide bridge formation between two thiol groups and the formation of a thiol-Au bond between a thiol group and a gold surface, preferably the support is aldehyde derivatised silicon or quartz and is attached via a Shiffs bond to a lysine amine or the N-terminal amine of L. In a further aspect, the support is aldehyde derivatised silicon and is attached via a Shiffs bond to a lysine amine of L. Other linker-support attachment methods include covalent coupling via e.g. ester bonds, amide bonds, as well as non-covalent coupling such as ionic bonding and hydrophobic interactions.

In one aspect of the invention, the support is insoluble. In a further aspect of the invention the support comprises a material selected from the group consisting of polymer such as polystyrene, polyethylene, polyester, polyethermide, polypropylene, polycarbonate, polysulfone, polymethylmethacrylate, or poly(vinylidene fluoride), and silicon or quartz. In still a further aspect the support is selected from the group consisting of gold, electronic chip, slide, wafer, resin, well, tube, micro array and membrane. In still a further aspect the support is selected from the group consisting of an electronic chip, slide, wafer, resin, well, tube, micro array and membrane. In another aspect the support comprises a material selected from the group consisting of polystyrene, polyethylene, polyester, polyethermide, polypropylene, polycarbonate, polysulfone, polymethylmethacrylate, poly(vinylidene fluoride), and silicon.

In another aspect of the invention, the insoluble support may be coated with a layer of the disulphide-containing linker.

In one aspect of the invention, a carrier comprising a support attached to at least one disulphide-containing linker as defined herein, is provided. In a further aspect of the invention, the support is insoluble.

The coupled carrier according to the invention is obtainable by the method according to the invention.

In one aspect of the invention, the polypeptide coupled to the carrier is selected from the group consisting of an enzyme such as selected from the group consisting of cutinase, chymosin, glucose oxidase, lipase, lysozyme, alkaline phosphatase and plasminogen, transcription factor, protein domain, binding protein, antigen and immunoglobulin, such as a F(ab) fragment.

The carrier comprising the support to which at least one disulphide-containing linker is attached is preferably insoluble. In one aspect of the invention, the carrier comprises a support which is selected from the group consisting of an electronic chip, slide, wafer, resin, well, tube, micro array and membrane. In a further aspect of the invention, the support comprises a material selected from the group consisting of polystyrene, polyethylene, polyester, polyethermide, polypropylene, polycarbonate, polysulfone, polymethylmethacrylate, poly(vinylidene fluoride), and silicon.

In another aspect, the invention relates to an insoluble coupled carrier comprising one or more polypeptide(s) coupled by the method according to the invention. The coupled polypeptide may suitably be specifically oriented. The coupled carrier according to the invention comprises a support such as one selected from the group consisting of an electronic chip, slide, wafer, resin, well, tube, micro array and membrane and the material of the support may be selected from but is not limited to the above group consisting of polystyrene, polyethylene, polyester, polyethermide, polypropylene, polycarbonate, polysulfone, polymethylmethacrylate, poly(vinylidene fluoride), and silicon.

The polypeptide coupled to the carrier is preferably spatially controlled.

In one aspect of the invention, the coupled carrier is used in a bio-functional reaction such as a bio-sensor, chromatography, immunodetection, enzyme assay, nucleotide binding detection, protein-protein interaction, protein modification, carrier targeting and protein targeting. The coupled carrier may also be used in a diagnostic or biosensor kit.

In one aspect of the invention, the polypeptide may be released from the carrier by irradiating the polypeptide to create a thiol group in the polypeptide be disulphide bridge disruption.

In another aspect of the invention, the use of the method according to the invention for the production of polypeptide-based surface coating is provided, in particular for use in the production of polypeptide-based biosensors, polypeptide-based micro arrays, and food packing materials with polypeptide-based surface coatings e.g. for the production of anti-microbial food packing materials.

As used herein, the term “biosensor” comprises an analytical devise incorporating biological or biologically-derived sensing elements, such as an amino acid (e.g., cysteine), protein, antibody, nucleic acid, microorganism or cell. The sensing element is either integrated within or intimately associated with a physicochemical transducer. The general aim of a biosensor is to produce either discrete or continuous signals that are proportional to a single analyte or a related group of analytes such as e.g. digital electronic signals or light signals.

The coupled carrier according to the invention may suitably be used for drug delivery.

In one aspect of the invention, a method of delivering a drug or prodrug to a patient is provided, comprising the following steps of:
providing a carrier coupled to one or more polypeptides
administering the carrier-coupled polypeptide to a patient
irradiating the carrier-coupled polypeptide to create a thiol group in the molecule by disulphide bridge disruption, and thereby releasing the polypeptide from the carrier.

In the above method of the invention, the carrier in itself may be a pharmaceutical drug and in another aspect of the invention, the coupled polypeptide is a drug or a prodrug.

As used herein, the term ‘pharmaceutical drug’ comprises articles intended for use in the diagnosis, cure, mitigation, treatment, or prevention of disease in man or other animals; and articles (other than food) intended to affect the structure or any function of the body of man or other animals and articles intended for use as a component of any article specified above.

The method of coupling to a carrier according to the invention can be used to construct various types of disulphide-linked oligomers or polymers. In one aspect of the invention light-induced thiol groups in a given protein or peptide or other biomolecules can be coupled to a carrier. Provided that the concentration of protein and carrier molecules in the coupling reaction is sufficiently high, SS based cross-linking between neighbouring molecules will take place. While the light-induced protein in one aspect of the invention should contain an SS bridge, the possession of an aromatic residue as close spatial neighbour is not essential, since the aromatic contribution to the reaction may be supplied by aromatics residues, or compounds that can mimic them, added to the coupling reaction.

In a further aspect of the invention the method of light-induced coupling to a carrier can be usefully applied to other types of carrier molecule, such as pharmaceutical drugs, in order to facilitate their effective delivery. For example, light-induced thiol coupling of a water-soluble molecule containing a disulphide bridge (including but not limited to a peptide or protein) to a drug can help the solubilisation and delivery of water-insoluble, poorly soluble or hydrophobic drugs. Furthermore, the molecule coupled to the drug may serve to protect the drug from its physiological environment, and hence improve its stability in vivo. This particular feature makes this technology attractive for the delivery of labile drugs such as proteins. Localized delivery of the molecule-coupled drug, by implantation at the site of treatment, would reduce systemic exposure of the patient to the drug. Carrier-linked prodrugs are generally defined as prodrugs that contain a temporary linkage of a given active substance to a transient carrier group that produces improved physicochemical or pharmacokinetic properties and that can be easily removed in vivo, usually by a hydrolytic cleavage. In an aspect of the present invention, light-mediated disruption of the disulphide bond linking a drug to a molecule can be used to achieve a controlled release of the active drug from the molecule-coupled form, implanted in the patient. This would minimise the frequency of drug delivery to the patient, and provide for light-controlled dosing. The process of drug delivery may be optimised, by only illuminating those regions of the body where drug release is necessary. These features would improve patient compliance, especially for drugs used for chronic indications, requiring frequent injections (such as for deficiency of certain proteins or metabolites). Controlled drug release could be induced by infra-red light (via two-photon excitation) in the case of transdermal drug delivery, within the penetration range of infra-red light, while the greater penetration of UV light (or infra-red light via three-photon excitation) would facilitate drug release deeper within the patient. Also, the use of optical fibers allows the delivery of light at various depths in the body, as used in PDT (photodynamic therapy), as for example in the treatment of cancer/tumor patients. Since a solvent exposed disulphide bridge will be broken in a reducing environment, the drug could also be released when the carrier coupled with the drug has entered a reducing environment such as the cytoplasmic space of a cell.

The method according to the invention of light induced thiol coupling can also be used to immobilise a protein on a support. The disulphide bond between the linker and the protein is stable, and extensive washing after immobilisation will not displace the protein. The density of proteins on a support can be controlled by varying the protein concentration, or the intensity and duration of UV-irradiation, and subsequently blocking the remaining activated thiol groups on the surface with reagents such as L-cysteine, (2-(2-pyridinyldithio)ethaneamine hydrochloride (PDEA) or with a thiol-lipid bilayer (Hong Q., et. al., 2001, Biochemical Society Transactions 29(4):587-582). The support, with evenly distributed immobilised proteins, is therefore in this aspect blocked to prevent non-specific binding. According to this aspect of the present invention, the method of immobilisation does not involve any chemical steps, since the thiol-activated proteins formed by UV radiation, can spontaneously self-assemble on the support. The described thiol and disulphide exchange reactions are an effective and rapid way to bind molecules to carriers.

Immobilisation of a polypeptide on a carrier can also be spatially controlled. Present day laser technology allow for focal spots with dimensions of 1 micrometer or less. If a specific polypeptide or target molecule, containing SS bridge(s), is incubated with the disulphide-containing carrier the light-induced thiol group formation and coupling could be limited to the focal points of illumination. This approach would allow for an extremely dense packing of identifiable and different molecules on a carrier surface. Thus, the method of the present invention could be used for charging micro arrays with molecules.

In a further aspect of the present invention, the orientation of the immobilised polypeptide can be controlled in a uniform and reproducible manner. Prolonged selective excitation of e.g. tryptophan residues in a polypeptide such as a protein will only lead to disruption of those disulphide bridges to which excitation energy is transferred. The location of these photo-disruptable disulphide bridges, forming free thiol groups, can be predicted from the protein's structure, where it is known from three-dimensional models, nuclear magnetic resonance (NMR) or X-ray diffraction crystallography analysis. In those cases where only a single thiol group is induced by irradiation of tryptophan residues in a protein, as is the case for cutinase, then immobilisation of the protein on a support will occur exclusively via this thiol group. In contrast to alternative methods of disulphide bridge disruption, the light-induced method in one aspect of the present invention may lead to targeted disruption of disulphide bridges forming one or only a few accessible thiol groups, whose position can be precisely predicted. The subsequently immobilised proteins will thus have a single or very limited number of orientations. Since e.g. cutinase has only a single thiol group for immobilisation, which is distant from the active site, the accessibility of substrates will not be limited by immobilisation. Immobilisation via a surface accessible thiol group, remote from the protein's active site, as is the case for cutinase and lysosyme, is also less likely to alter the conformation or structural properties of the protein. In other words, the immobilisation method of the present invention serves to preserve the native state of the immobilised protein. All functional/structural assays performed on proteins, which are immobilised in a uniform orientation according to the methods of the present invention, can generate data derived from a uniform population of proteins. The structural and functional uniformity of the immobilised proteins, and retention of their native state, is of primary importance for screening or assaying proteins for catalytic, binding, or any other biological properties and provides one of the many valuable advantages of the present invention. In the case of proteins such as lysosyme, which have anti-microbial properties, it is useful to be able to immobilise said proteins on a surface (e.g. food, skin, packaging) in order to prevent microbial growth and infection.

In a further aspect of the present invention, the bond immobilising the protein to the carrier may be disrupted, releasing the protein into solution. Disulphide bridges between a protein and the carrier can be disrupted e.g. with UV irradiation, in the same way as disrupting a disulphide bond e.g. on a protein, where an aromatic amino acid is a spatial neighbour. In one aspect of the invention, the aromatic amino acid is either located on the immobilised protein itself, or is supplied in the form of a solution of an aromatic amino acid, such as tryptophan, applied to the support surface. Disulphide bonds (SS) themselves are known to be disrupted by approximately 254 nm light. Alternatively, disulphide bridges between a protein and a carrier can be disrupted with (dithiothreitol) DTT, or other reducing agents known to persons skilled in the art. Following disruption of the immobilisation bond, the released protein can be purified, if necessary, and used in further experiments.

A further aspect of the present invention is regenerating a gold surface by removing proteins that are immobilised through a thiol-Au bond with O₂-plasma treatment or Piranha, thereby removing the top layer of the gold surface including the proteins.

In a further aspect of the invention, the polypeptide can be released from the carrier by irradiating the polypeptide to create a thiol group in the polypeptide by disulphide bridge disruption.

The features disclosed in the foregoing description may, both separately and in any combination thereof, be material for realising the invention in diverse forms thereof.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

The following formulation examples are illustrative only and are not intended to limit the scope of the invention in any way.

### EXPERIMENT

Lysozyme was used as a model protein. Lysozyme was immobilised on a disulphide-derivatised quartz carrier (sensor) surface. A disulphide-peptide was designed with a lysine at the N-terminus (which allows chemical attachment to an aldehyde-derivatised surface via a Shiffs bond between the Lysine amine and/or the N-terminal amine and the surface aldehyde) and a disulphide-ring in the C-terminus (the C-terminus is amidated in order to prevent reactions between the C-terminal carboxyl group and the protein). A tryptophan is located in close proximity of the disulphide bridge which allows for UV-induced disruption of the disulphide bridge, which then leads to the formation of free thiols which can react with free thiols in the protein that is to be immobilised.

### Chemicals

25 mM Tris buffer pH 7.0 prepared from Trizma base (Sigma T-6066, lot.033K5405). Detergent in phosphate buffered saline (PBS buffer) pH 7.4 was made from a 10×PBS stock (Fluka 79383). Tris-detergent and PBS-detergent solutions were prepared by addition of 0.1 v/v % polyoxyethylenesorbitan monolaurate (Sigma P7949/Tween20) to the respective buffers.

Lysozyme (Sigma L-7651) was prepared as 2 μM solutions in 25 mM Tris buffer at pH 7.0.

A synthetic peptide was purchased as a request order from KJ Ross-Petersen ApS (www.ross.dk). The peptide sequence is KAMHAWGCGGGC-NH2 (cf. SEQ ID NO 9) where CGGGC is cyclic, i.e. it is closed with a disulphide bridge between the two cysteines (C). The molecular mass of this peptide is ˜1175 g/mole. The peptide was dissolved to stock solutions in 25 mM Tris buffer at pH 7.0 of approximately 250 μM peptide and diluted to 10 μM prior use.

### Surface Preparation of TIRF Quartz Slides

Chemical modification of glass, quartz and metal oxide surfaces can be performed by silanization. Silane chemistry allows functionalization of a quartz surface with different active groups, such as free aldehyde groups, by covalently linking Si to the quartz OH-groups.

Prior to silanization, the slides were cleaned by immersing them into 70-75° C. chromosulphuric acid (Merck 1.02499) for 1 hour, and rinsed thoroughly in deionized water at ambient room temperature. Then the slides were hydroxylated in order to increase the number of OH groups at the surface. Hydroxylation was performed by immersing the slides for 1 hour into 99-100° C. 5 w/v % potassium persulphate (K2S2O8 99%, Acros Organics 20201) in deionised water. After hydroxylation the slides were flushed with deionized water and dried rapidly (using dry compressed air).

In order to prepare the quartz surface for peptide derivatisation, the quartz slides were coated with an aldehyde base linker. Aldehyde-coating was performed by applying 350 μl 0.3% v/v Triethoxysilylbutyr-aldehyde (Gelest SIT8185/5C-6406, purchased from ABCR, Germany, www.abcr.de) in m-xylene (99+%, Acros Organics 1808600100) on each quartz slide (12 cm2). The slides were incubated in a closed glass Petri dish (Ø=14 cm) at ambient room temperature (20-25° C.). Subsequently, the surface was flushed with pure xylene, 70% ethanol and deionized water. Finally, the slides were dried using compressed dry air.

### UV-Assisted Immobilisation Using TIRF

### Instrumental Setup

A TIRF system (BioElectroSpec, Inc., Harrisburg, Pa.) coupled with a fluorescence spectrophotometer (PTI QM-2000 from Photon Technology Int., Lawrenceville, N.J.) with a 75W Xenon arc source was used. The PTI instrument excitation and emission slits were set at 6 nm. The flow chamber was comprised by a “sandwich” consisting of a TIRF quartz prism, a TIRF quartz slide (with the sample surface), a 10 μm polyureathane gasket (comprising the chamber thickness), and a back block with holes to channel the solution through the flow chamber. In this setup, the excitation light travels through the prism and slide, hits the interface between slide and solution, leading to total internal reflection at the interface. The reflected beam generates an evanescent electromagnetic field in the centre of the TIRF slide—this area of the TIRF slide serves thus as the sensor surface. Only fluorophores that are present at the surface and in close proximity of the surface (within the evanescent field) are excited and fluoresce.

The TIRF quartz prism and the TIRF quartz slide were sandwiched using glycerol in between since glycerol and quartz have almost equal refractive indices and allow therefore for light passage with minimal interference.

In order to analyze the amount of immobilised protein, the sample in the flow chamber was excited at 295 nm and the fluorescence emission intensity was monitored at 350 nm (tryptophan excitation and emission). In order not to UV-damage the surface aldehyde coating and the samples in the TIRF chamber, the excitation light was only on for 3 seconds whenever a data points was acquired. Both the excitation slit and the manual slit of the lamp house were closed in order to exclude light at all other time points. During UV-assisted immobilisation, the lamp was on for the necessary time, i.e. from a few seconds to minutes.

The flow rate was 0.25 ml/min during loading of peptide and protein, while the rinsing flow rate was 1.75 ml/min.

### Peptide Coating of Carrier and Protein Immobilisation

The aldehyde-coated slide was attached on the TIRF quartz prism as described above. The TIRF flow-chamber was assembled and mounted vertically in the instrument. As mentioned above, the fluorescence intensity emission due to tryptophan excitation was used as a measure of peptide and protein immobilisation (since both the peptide and the protein contain tryptophans).

Experiments were performed as follows:

| Start time | |
|---|---|
| (sec) | Action |
| 0000 | Buffer rinse |
| 0050 | Measurement |
| 0290 | Measurement |
| 0300 | Peptide (1.25 ml at 0.25 ml/min) |
| 1500 | 0.1% Tween20/25 mM Tris pH 7.0 |
| 1700 | Buffer rinse |
| 1850 | Buffer rinse |
| 1990 | Measurement/Light on continuously |
| 2000 | Protein (0.25 ml at 0.25 ml/min) |
| 2085 | Light off |
| 2120 | 0.1% Tween20/PBS pH 7.4 |
| 2300 | Buffer rinse |
| 2450 | 0.1% Tween20/PBS pH 7.4 |
| 2600 | Buffer rinse |
| 2750 | Measurement |
| 2800 | 0.1% Tween20/PBS pH 7.4 |
| 2950 | Buffer rinse |
| 3100 | 0.1% Tween20/PBS pH 7.4 |
| 3250 | Buffer rinse |
| 3400 | Measurement |
| 3450 | 0.1% Tween20/PBS pH 7.4 |
| 3600 | Buffer rinse |
| 3700 | Measurement |

Volume and flow rate was 2.50 ml and 1.75 ml/min unless stated otherwise.

Sample was only excited 2-3 seconds during readings and continuously during UV-immobilisation of the protein. At all other time points, the light shutters were closed. The control experiment was equal to the procedure above, except that light was shut off during protein loading.

No immobilization was observed where the protein was not exposed to UV light.

When comparing above experiment to an experiment using a SH-coated surface there was significant immobilization without UV light.

1. A method of coupling a polypeptide to a carrier via at least one disulphide bond, said carrier comprising a support wherein said support is attached to at least one disulphide-containing linker capable of being activated by irradiation to contain reactive thiol groups, wherein said method comprises the following steps of:
a. incubating a polypeptide containing at least one reactive thiol group with the carrier,
b. irradiating the carrier to create reactive thiol groups,
or
a. irradiating a polypeptide containing at least one disulphide bridge to create reactive thiol groups in the polypeptide by disulphide bridge disruption,
b. irradiating the carrier to create reactive thiol groups, and
c. incubating the irradiated polypeptide with the irradiated carrier, wherein step a and step b can be simultaneous or sequential in any order.

2. The method according to claim 1, wherein said disulphide-containing linker is a peptide linker comprising at least one amino acid.

3. The method according to claim 2, wherein said peptide linker comprises at least one aromatic amino acid.

4. The method according to claim 3, wherein said peptide linker has formula I-L-D (formula I), wherein L is attached to the support and comprises at least one amino acid which is different from an amino acid which is capable of being activated by irradiation to contain at least one reactive thiol group and which does not contain a reactive thiol, and D is a non-cyclic sequence of amino acids or a cyclic sequence of amino acids, which non-cyclic or cyclic sequence comprises at least two cysteines (C) covalently joined by a disulphide bridge and wherein one of the cysteine (C) is bound to L.

5. The method according to claim 4, wherein L comprises 1-30 amino acids.

6-7. (canceled)

8. The method according to claim 4, wherein D comprises 2-30 amino acids.

9-10. (canceled)

11. The method according to claim 4, wherein D is a cyclic sequence of amino acids.

12. The method according to claim 4, wherein D has the following sequence C(X)ₙC, wherein X independently is any amino acid which does not comprise a reactive thiol group, n is from 1 to 10 and the two cysteines (C) are covalently joined by a disulphide bridge.

13. (canceled)

14. The method according to claim 4, wherein D has the following sequence CC(Xi)₀₁, wherein Xi independently is any amino acid which does not comprise a reactive thiol group, n1 is from 0 to 10, and the two cysteines (C) are covalently joined by a disulphide bridge and a peptide bond.

15. The method according to claim 4, wherein D has the following formula C-S-S-C(Xa)₀₁, wherein X₁ independently is any amino acid which does not comprise a reactive thiol group, n1 is from 0 to 10, and the two cysteines (C) are covalently joined by a disulphide bridge.

16. (canceled)

17. The method according to claim 4, wherein L comprises one or more aromatic amino acids.

18. The method according to claim 17, wherein L comprises an aromatic amino acid separated from the cysteine (C) in D bound to L by at least one amino acid.

19. The method according to claim 18, wherein the aromatic amino acid is tryptophan.

20. The method according to claim 4, wherein L has the following sequence (X₃)ₙ₃W(X₄)ₙ₄, wherein X₃ and X₄ independently are any amino acid which does not comprise a reactive thiol group, W is tryptophan, and n3 and n4 independently are from 1 to 5.

21. The method according to claim 4, wherein L-D has the following sequence K(X₅)ₙ₅WX₆CGGGC, wherein X₅ and X₆ independently are any amino acid which does not comprise a reactive thiol group, W is tryptophan, K is lysine, n5 is 3, G is glycine, and the two cysteine molecules (C) are covalently joined by a disulphide bridge.

22. The method according to claim 4, wherein L-D has the following sequence KAMHAWGCGGGC-NH2 (SEQ ID NO: 9), wherein CGGGC (SEQ ID NO: 8) is cyclic and the two cysteine molecules (C) are covalently joined by a disulphide bridge, K is lysine, A is alanine, M is methionine, H is histidine, W is tryptophan, and G is glycine.

23. The method according to claim 4, wherein L-D has the following formula KAMHAWGC-S-S-CX₇X₈—NH2 (SEQ ID NOS: 10 and 12), wherein X₇ and X₈ independently are any amino acid which does not comprise a reactive thiol group, and the two cysteine molecules (C) are covalently joined by a disulphide bridge, K is lysine, A is alanine, M is methionine, H is histidine, W is tryptophan, and G is glycine.

24. The method according to claim 4, wherein L-D has the following formula KAMHAWGC-S-S-CGG-NH2 (SEQ ID NOS: 10 and 11), wherein the two cysteine molecules are covalently joined by a disulphide bridge K is lysine, A is alanine, M is methionine, H is histidine, W is tryptophan, and G is glycine.

25. The method according to claim 2 wherein said peptide linker has formula II -D (formula II), wherein D is as defined in claim 4.

26. The method according to claim 4, wherein D comprises an aromatic amino acid.

27. The method according to claim 1, wherein the support is insoluble.

28. The method according to claim 1, wherein the support is coated with a layer of the disulphide-containing linker.

29-31. (canceled)

32. The method according to claim 1, wherein the free thiols in said polypeptide are formed after chemical treatment.

33. The method according to claim 1, wherein the polypeptide comprises one disulphide bridge.

34. The method according to claim 1, wherein said irradiation step comprises light of a wavelength that excites one or more aromatic amino acids.

35. The method according to claim 1, wherein said irradiation step comprises light of a wavelength that excites one specific aromatic amino acid.

36. The method according to claim 1, wherein the polypeptide comprises one or more aromatic amino acid(s) selected from the group consisting of tryptophan, tyrosine and phenylalanine.

37. The method according to claim 1, wherein the irradiation is performed by multi-photon excitation.

38. The method according to claim 1, wherein said irradiation comprises light with a wavelength of about 250-320 nm.

39-42. (canceled)

43. The method according to claim 36, wherein said polypeptide is irradiated in the presence of a free aromatic amino acid.

44. The method according to claim 1 further comprising the steps of:
a. verifying the presence of one or more disulphide bridges in said polypeptide,
b. identifying one or more aromatic amino acid residues in close spatial proximity to said one or more disulphide bridges, and
c. selecting a wavelength which specifically excites one or more of said aromatic amino acid residues, thereby disrupting one or more of said disulphide bonds.

45. The method according to claim 44, wherein said immobilisation is spatially controlled.

46. The method according to claim 44, wherein said polypeptide may be released from the carrier by irradiating the polypeptide to create a thiol group in the polypeptide by disulphide bridge disruption.

47-48. (canceled)

49. A carrier comprising a support according to claim 30, wherein said carrier is attached to at least one disulphide-containing linker as defined in claim 1.

50-52. (canceled)

53. The carrier according to claim 1 wherein said carrier is coupled to one or more polypeptides.

54-66. (canceled)
